(19) United States
(12) Patent Application Publication
(10) Pub. No.: US 2008/0294230 A1
(43) Pub. Date: Nov. 27, 2008

(54) Apparatus and methods for deploying self-expanding stents

(75) Inventor: Fred T. Parker, Unionville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/805,803

(22) Filed: May 24, 2007

(51) Int. Cl. A61F 2/06

(52) U.S. Cl. 623/1.11

(57) ABSTRACT

Apparatus and methods are provided for improved deployment of self-expanding stents. One advantage of the improved delivery system is that energy storage within a portion of an outer sheath and/or an inner tube may be reduced during the deployment of the stent. In a first embodiment, the outer sheath and the inner tube may be coupled together using a plurality of engaging threaded members, such that circumferential rotation of the inner tube with respect to the outer sheath retracts the outer sheath to deploy the stent. In an alternative embodiment, a fluid reservoir may be provided between the inner tube and the outer sheath. A proximal sealing ring may be disposed annularly between the inner tube and the outer sheath, such that when the fluid reservoir is filled, the proximal sealing ring is urged proximally to engage and retract the outer sheath. Using these techniques, energy build-up in the outer sheath and/or inner tube may be substantially reduced and improved accuracy in deploying the stent may be achieved.

### BACKGROUND

The present invention relates generally to medical devices, and more particularly, to apparatus and methods for improved deployment of self-expanding stents.

Atherosclerosis and other occlusive diseases are prevalent among a significant portion of the population. In such diseases, atherosclerotic plaque forms within the walls of the vessel and blocks or restricts blood flow through the vessel. Atherosclerosis commonly affects the coronary arteries, the aorta, the iliofemoral arteries and the carotid arteries. Several serious conditions may result from the restricted blood flow, such as ischemic events.

Various procedures are known for treating stenoses in the arterial vasculature, such as the use of atherectomy devices, balloon angioplasty and stenting. Stenting involves the insertion of a usually tubular member into a vessel, and may be used alone or in conjunction with an angioplasty procedure. Stents may be balloon expandable or self-expanding. If the stent is balloon expandable, the stent typically is loaded onto a balloon of a catheter, inserted into a vessel, and the balloon is inflated to radially expand the stent. Self-expanding stents typically are delivered into a vessel within a delivery sheath, which constrains the stent prior to deployment. When the delivery sheath is retracted, the stent is allowed to radially expand to its predetermined shape.

One problem that exists with conventional self-expanding stent deployment systems is that the longitudinal force imposed upon the delivery sheath can be relatively high. Typically, an inner tube disposed proximal to the stent is held steady to longitudinally restrain the stent while a proximal end of the delivery sheath is retracted, thereby exposing the stent. However, as the proximal end of the delivery sheath is being pulled, a significant build-up of energy may occur along the length of the delivery sheath due to friction between the delivery sheath and the stent. In particular, the act of deployment typically imposes a stretch on the overall length of the delivery sheath, and thus, results in a substantial axial compressive force on the overall length of the inner tube. The stored energy in the delivery sheath and/or inner tube may be suddenly released, causing the stent to move forward unexpectedly, i.e., “jump” forward, leading to inaccurate placement of the stent in a vessel.

Moreover, the significant forces imposed upon the delivery sheath containing the self-expanding stent, and/or the inner tube disposed proximal to the stent, may lead to various system failures. For example, the delivery sheath itself may be stretched beyond its maximum ability and may not recover elasticity or may break in half, various fittings may become disengaged due to the forces imposed, the inner tube may become overly compressed into an “accordion” shape, and so forth.

Problematically, the energy build-up within the delivery sheath and inner tube may be even more affected as the length of the delivery system is increased. Since relatively long self-expanding stents, e.g., having lengths between 200 to 300 mm, may become prevalent in newer devices, the problem of energy build-up in the delivery sheath and inner tube may become a larger concern. Accordingly, there is a need for improved delivery systems for self-expanding stents.

### SUMMARY

The present invention provides apparatus and methods for improved deployment of self-expanding stents and may reduce the energy storage within a portion of an outer sheath and/or an inner tube of the delivery system during deployment of the stent.

In a first embodiment, an inner tube is disposed substantially coaxially inside of an outer sheath, and a self-expanding stent is disposed in a compressed state within the outer sheath at a location distal to the inner tube. At least one threaded member is coupled to the outer sheath, and at least one mating threaded member is formed on an outer surface of the inner tube. In operation, circumferential rotation of the inner tube with respect to the outer sheath retracts the outer sheath to deploy the stent. By using a threading engagement between the outer sheath and the inner tube, the longitudinal forces and energy storage imposed upon the outer sheath and the inner tube may be substantially reduced, relative to techniques that rely on pulling on a proximal end of the outer sheath to retract the sheath. Moreover, the outer sheath may not be exposed to substantial stretching, and the inner tube may not be exposed to substantial compression, which may result in a more accurate deployment of the self-expanding stent.

In an alternative embodiment, the apparatus comprises an inner tube disposed substantially coaxially inside of an outer sheath, and a self-expanding stent is disposed in a compressed state within the outer sheath at a location distal to the inner tube. At least one fluid reservoir is disposed between the inner tube and the outer sheath, and at least one lumen is in fluid communication with the fluid reservoir. During use, the delivery of fluid to the fluid reservoir via the lumen is adapted to impose a pressure upon the outer sheath to retract the outer sheath and permit deployment of the self-expanding stent.

In the latter embodiment, the fluid reservoir may comprise proximal and distal sealing rings. The distal sealing ring may be disposed annularly between the inner tube and the outer sheath within a distal section of the fluid reservoir. The proximal sealing ring may be disposed annularly between the inner tube and the outer sheath within a proximal section of the fluid reservoir. The outer sheath may comprise a step disposed adjacent to the proximal sealing ring. When fluid fills the reservoir, the distal sealing ring cannot move distally, but the proximal sealing may be incrementally advanced proximally over the inner tube to push against the step in the outer sheath, thereby causing retraction of the outer sheath with respect to the inner tube. Using this technique, the longitudinal forces and energy storage imposed upon the outer sheath and the inner tube may be substantially reduced, and a more accurate deployment of the self-expanding stent may be achieved.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side-sectional view of a distal region of an apparatus that may be used to deploy a self-expanding stent.

FIG. 2 is a side-sectional view illustrating enlarged features of the apparatus of FIG. 1.

FIG. 3 is a side-sectional view of a distal region of an alternative apparatus that may be used to deploy a self-expanding stent.

FIG. 4 is a side-sectional view illustrating enlarged features of the apparatus of FIG. 3.

FIG. 5 is a side-sectional view of a distal region of a further alternative apparatus that may be used to deploy a self-expanding stent.

FIG. 6 is a side-sectional view illustrating enlarged features of the apparatus of FIG. 5.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patient's anatomy during a medical procedure.

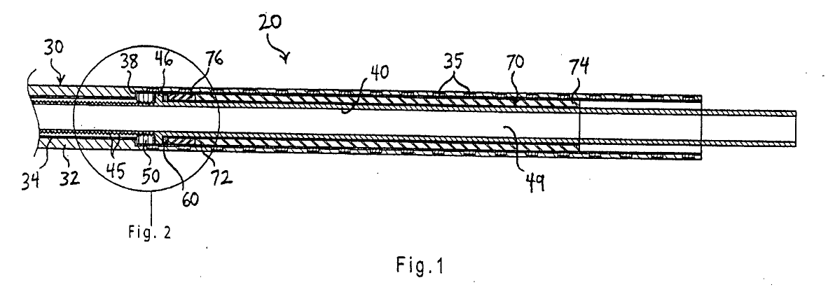

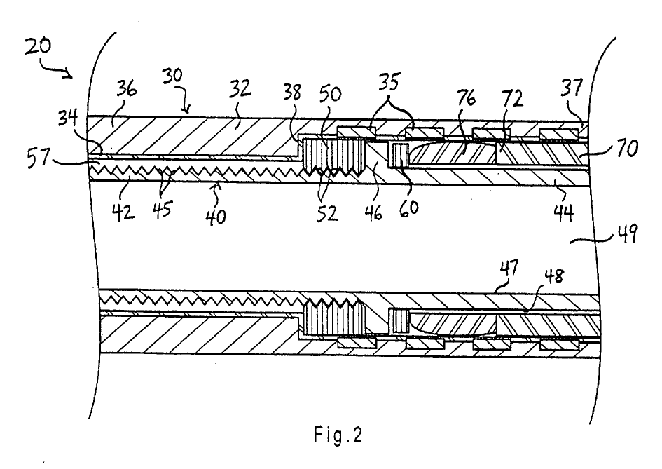

Referring now to FIGS. 1-2, a first embodiment of an apparatus for deploying a self-expanding stent is described. Apparatus 20 comprises outer sheath 30, inner tube 40, and at least one self-expanding stent 70. As will be explained further below, energy build-up associated with the retraction of outer sheath 30 with respect to inner tube 40 may be limited to an area substantially in the vicinity of stent 70, and may not span a significant portion of the overall length of the outer sheath and/or the inner tube.

As shown in FIG. 2, outer sheath 30 has proximal and distal regions 36 and 37 may comprise outer member 32 and inner member 34. Outer and inner members 32 and 34 may be disposed substantially adjacent to one another. A coil member 35, such as a flat steel coil, may be sandwiched between outer and inner members 32 and 34 along distal region 37, as depicted in FIGS. 1-2. One advantage of an outer sheath 30 having this type of construction is that the provision of coil member 35 may reduce the likelihood of stent 70 catching upon outer sheath 30 upon retraction of outer sheath 30 due to the provision of coil member 35.

In one embodiment, inner member 34 may comprise a layer of polytetrafluoroethylene (PTFE), while outer member may comprise nylon. As will be apparent, other materials may be employed. Further, in alternative embodiments, inner member 34 and/or coil member 35 may be omitted, i.e., outer sheath 30 may comprise a tubular material comprising one or two layers, with or without coil member 35 embedded at its distal region.

As shown in FIG. 2, step 38 may be disposed between proximal and distal regions 36 and 37, thereby making a thickness of proximal region 36 greater than a thickness of distal region 37. By reducing the thickness of distal region 37, stent 70 may be accommodated without substantially increasing the overall profile of apparatus 20.

Inner tube 40 may be disposed in a coaxial arrangement with outer sheath 30, as shown in FIGS. 1-2. Inner tube 40 comprises proximal and distal regions 42 and 44, with outwardly-protruding step 46 formed therebetween. Inner tube 40 further comprises inner and outer surfaces 47 and 48. Along proximal and distal regions 42 and 44, inner surface 47 is substantially smooth to permit advancement of medical components through lumen 49. Along a portion of proximal region 42, outer surface 48 comprises a plurality of threaded members 45. The threaded members 45 preferably are not disposed along distal region 44, as shown in FIG. 2.

Apparatus 20 may further comprise block member 50, which has an outer surface attached to inner member 34 of outer sheath 30, and further has an inner surface comprising threaded members 52. In the embodiment depicted in FIGS. 1-2, threaded members 52 of block member 50 are adapted to engage threaded members 45 of inner tube 40, as explained in greater detail below. While block member 50 is depicted as being a separate component from outer sheath 30, in an alternative embodiment block member 50 may be formed integrally with outer sheath 30 such that threaded members 52 are formed within a portion of inner member 34. Preferably, a small annular passageway 57 is formed between a portion of outer sheath 30 and inner tube 40 to reduce potential friction between threaded members 45 of inner tube 40 and inner member 34.

Apparatus 20 may also comprise at least one washer 60 disposed annularly between distal region 44 of inner tube 40 and distal region 37 of outer sheath 30 at a location proximal to stent 70, as shown in FIGS. 1-2. Washer 60 may reduce the likelihood of inadvertently circumferentially rotating stent 70 while inner tube 40 is rotated with respect to outer sheath 30, as explained in further detail below.

Stent 70 comprises proximal and distal ends 72 and 74. Various types of self-expanding stents 70 may be used in conjunction with the present invention. For example, stent 70 may be made from numerous metals and alloys, including stainless steel, nitinol, cobalt-chrome alloys, amorphous metals, tantalum, platinum, gold and titanium. Stent 70 also may be made from non-metallic materials, such as thermoplastics and other polymers. The structure of stent 70 may also be formed in a variety of ways to provide a suitable intraluminal support structure. Stent 70 may generally comprise a zig-zag shape, i.e., formed from a single wire having a plurality of substantially straight segments and a plurality of bent segments disposed between the substantially straight segments. Alternatively, stent 70 may comprise any number of shapes, for example, made from a woven wire structure, a laser-cut cannula, individual interconnected rings, a pattern of interconnected struts, or any other type of stent structure that is known in the art.

In one embodiment, at least one eyelet 76 may be integrally formed with or attached to proximal end 72 of stent 70, as shown in FIGS. 1-2. Eyelet 76, which may be disposed adjacent to washer 60 during delivery of apparatus 20, may be used to carry a radiopaque marker therein. Alternatively, stent 70 may have radiopaque markers disposed at one or more other locations along its longitudinal length.

Regardless of the configuration of stent 70, it has a reduced diameter delivery state, generally shown in FIGS. 1-2, in which it may be advanced to a target location within a vessel, duct or other anatomical site. Stent 70 further has an expanded deployed state in which it may be configured to apply a radially outward force upon a vessel, duct or other target location, e.g., to maintain patency within a passageway. In the expanded state, fluid flow is allowed through a lumen of the stent. Optionally, a graft material may be coupled to an inner or outer surface of stent 70, or stent 70 may be interwoven through the graft material. As will be apparent, common examples of graft materials may include Dacron, polyester, expandable polytetrafluoroethylene (ePTFE), polytetrafluoroethylene (PTFE), fabrics and collagen. However, graft materials may be made from numerous other materials as well, including both synthetic polymers and natural tissues. One graft material that holds particular promise in certain applications is small intestine submucosa (SIS). As those in the art know, SIS material includes growth factors that encourage cell migration within the graft material, which eventually results in the migrated cells replacing the graft material with organized tissues. Further, in certain applications, it may also be helpful to impregnate or coat the optional graft and/or stent 70 with various therapeutic drugs that are well-known to those in the art.

In operation, apparatus 20 may be delivered into a patient's vessel using known techniques. For example, apparatus 20 may be advanced over a wire guide that has traversed the patient's anatomy. The wire guide may be disposed through lumen 49 of inner tube 40. The positioning of apparatus 20 may be performed using fluoroscopic guidance. Moreover, one or more of the components of apparatus 20 may comprise a radiopaque marker to facilitate positioning of the device. Preferably, at least one radiopaque marker is disposed on stent 70 to facilitate positioning of stent 70 at a desired location, for example, within a stenosed region of a vessel.

When the desired positioning is achieved, a proximal end of inner tube 40 may be rotated circumferentially with respect to outer sheath 40, thereby causing a controlled retraction of outer sheath 30 with respect to inner tube 40 via the threaded engagement between threaded members 45 and threaded members 52. The proximal end of inner tube 40 may be rotated manually, e.g., using a rotatable handle and measurement indicia. Alternatively, a motor, such as a programmable stepper motor, may be coupled to the proximal end of inner tube 40 to rotate inner tube 40 a predetermined amount with respect to outer sheath 30.

As outer sheath 30 is retracted longitudinally with respect to inner tube 40, distal end 74 of stent 70 is no longer radially constrained within outer sheath 30. As outer sheath 30 is further retracted proximally, the remainder of stent 70 is exposed and may self-expand in a radially outward direction to engage a target site.

During retraction of outer sheath 30, protruding step 46 of inner tube 40 prevents proximal movement of stent 70. Further, as noted above, the provision of washer 60 may reduce the likelihood of stent 70 twisting as inner tube 40 is rotated circumferentially. Finally, if flat coil member 35 is employed within outer sheath 30, it may reduce the likelihood of stent 70 catching upon outer sheath 30 during retraction of outer sheath 30.

Advantageously, using the threading engagement of outer sheath 30 and inner tube 40, the longitudinal forces and energy storage imposed upon outer sheath 30 and inner tube 40 may be substantially reduced, relative to techniques that rely on pulling on a proximal end of outer sheath 30 to retract the sheath. Using the threading engagement of FIGS. 1-2, energy storage may be substantially limited to a region in the vicinity of stent 70, and may not span a substantial portion of the overall length of outer sheath 30 and inner tube 40. Moreover, outer sheath 30 may not be exposed to substantial stretching, and inner tube 40 may not be exposed to substantial compression. Therefore, with less energy storage in outer sheath 30, stent 70 may be less likely to “jump” in a distal direction upon deployment. Accordingly, using apparatus 20, a more accurate deployment of self-expanding stent 70 may be achieved, and the likelihood of the delivery system malfunctioning may be reduced.

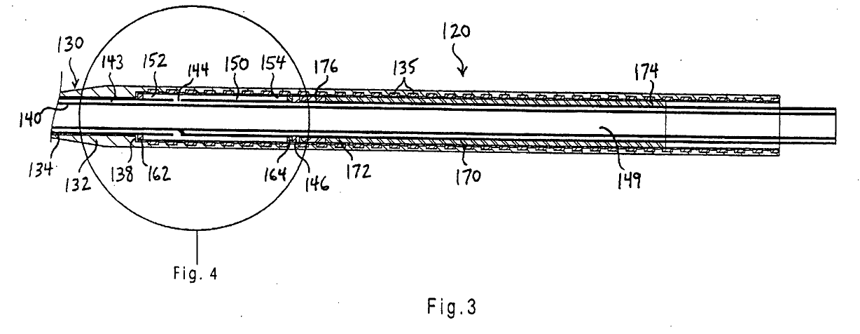

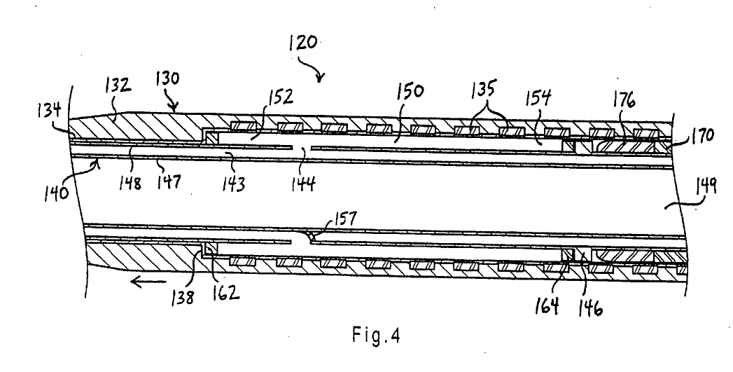

Referring now to FIGS. 3-4, an alternative embodiment is described. Apparatus 120 comprises outer sheath 130, inner tube 140, and at least one self-expanding stent 170. In the embodiment of FIGS. 3-4, outer sheath 130 may be provided substantially in accordance with outer sheath 30 of FIGS. 1-2, e.g., having inner and outer members 134 and 132, with coil member 135 embedded therein. Further, self-expanding stent 170 may be provided substantially in accordance with stent 70 of FIGS. 1-2, e.g., having at least one eyelet 176 disposed at the proximal end of the stent.

Inner tube 140 has proximal and distal regions, and further has inner and outer surfaces 147 and 148, respectively, as shown in FIG. 4. Lumen 143 may be concentrically disposed between inner and outer surfaces 147 and 148 and may span from the proximal region to the distal region of inner tube 140.

At least one fluid reservoir 150 is formed as a space between the outer surface 148 of inner tube 140 and inner member 134 of outer sheath 130, as shown in FIG. 4. One or more apertures 144 may be formed in outer surface 148 to provide fluid communication between lumen 143 of inner tube 40 and fluid reservoir 150. Fluid reservoir 150 may comprise proximal and distal reservoir sections 152 and 154, which are disposed proximal and distal to aperture 144, respectively, as shown in FIGS. 3-4.

Optionally, guiding element 157 may be disposed between inner and outer surfaces 147 and 148 of inner tube 140 and may be used to guide fluid from lumen 143 into fluid reservoir 150. If guiding element 157 is employed, a portion of inner tube 140 that is disposed distal to guiding element 157 may be solid, i.e., lumen 143 may terminate distal to guiding element 157. Alternatively, if guiding element 157 is omitted, fluid flowing through lumen 143 may flow partially into fluid reservoir 150 and partially through the entire length of inner tube 140 to exit the inner tube distal to apparatus 120.

Proximal and distal sealing rings 162 and 164 provide a substantially fluid tight seal for fluid reservoir 150. Proximal sealing ring 162 may be disposed axially between outer surface 148 of inner tube 140 and inner member 134 at a location proximal to aperture 144, as shown in FIG. 4. Similarly, distal sealing ring 164 may be disposed axially between outer surface 148 of inner tube 140 and inner member 134 at a location distal to aperture 144.

Any suitable fluid, such as saline, may be injected through lumen 143 into fluid reservoir 150. Further, any suitable material, such as polytetrafluoroethylene (PTFE), may be used in the manufacture of proximal and distal sealing rings 162 and 164.

In operation, apparatus 120 may be delivered into a patient's vessel in a manner described above with respect to apparatus 20 of FIGS. 1-2. When the desired positioning is achieved, fluid is injected through lumen 143 and into fluid reservoir 150. At this time, inner tube 140 may be held stationary.

As the fluid fills reservoir 150, pressure is imposed upon proximal and distal sealing rings 162 and 164. The pressure imposed upon distal sealing ring 164 tends to urge this sealing ring in a distal direction, however, since inner tube 140 is held stationary, distal sealing ring 164 pushes upon protruding step 146 of inner tube 140, and therefore cannot move distally. By contrast, the pressure imposed upon proximal sealing ring 162 urges proximal sealing ring 162 in a proximal direction. Since outer sheath 130 is not held stationary, the pressure urges sealing ring 162 proximally, which in turn presses upon step 138 of outer sheath 130 to urge outer sheath 130 proximally, as indicated by the arrow in FIG. 4. In effect, as the fluid fills reservoir 150, fluid flowing into proximal reservoir section 152 urges proximal sealing ring 162 and outer sheath 130 in a proximal direction, which in turn exposes stent 170 to enable deployment of the self-expanding stent.

Measurement indicia may be provided at the fluid source so that a physician may visually see how much fluid has been injected into fluid reservoir 150, which in turn may correlate to the amount that outer sheath 130 has been retracted proximally. By carefully controlling the injection of fluid into lumen 143 and reservoir 150, the physician may incrementally retract outer sheath 130 with respect to inner tube 140.

Advantageously, using the deployment system of FIGS. 3-4, the longitudinal forces and energy storage imposed upon outer sheath 130 and inner tube 140 may be substantially reduced, relative to techniques that rely on pulling on a proximal end of outer sheath 130 to retract the sheath. Moreover, outer sheath 130 may not be exposed to substantial stretching, and inner tube 140 may not be exposed to substantial compression. Therefore, with less energy storage in outer sheath 130, stent 170 may be less likely to “jump” in a distal direction upon deployment. Accordingly, using apparatus 120, a more accurate deployment of self-expanding stent 170 may be achieved, and the likelihood of the delivery system malfunctioning may be reduced.

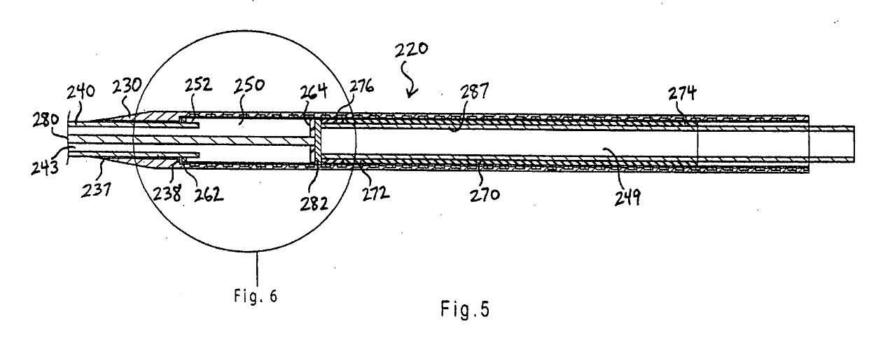

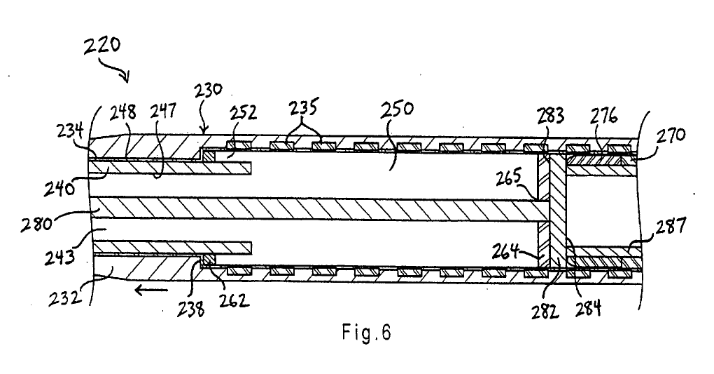

Referring now to FIGS. 5-6, an alternative to the embodiment of FIGS. 3-4 is described. In the embodiment of FIGS. 5-6, outer sheath 230 may be provided substantially in accordance with outer sheath 130, and self-expanding stent 270 may be provided substantially in accordance with stent 170 of FIGS. 3-4, e.g., having at least one eyelet 276 disposed at the proximal end of the stent. Apparatus 220 generally relies on the same principles as apparatus 120 of FIGS. 3-4, with some structural variations discussed below.

For example, apparatus 220 may comprise a central stylet 280 having proximal and distal ends. The distal end of stylet 280 may be attached to proximal surface 283 of disc member 282. Distal sealing ring 264 may comprise a central bore 265 to permit stylet 280 to be disposed therethrough, and further, distal sealing ring 264 may abut against proximal surface 283 of disc member 282, as depicted in FIG. 6.

Optionally, tubing 287 may be attached to distal surface 284 of disc member 282. Tubing 287 may be disposed annularly inside of stent 270 to thereby confine stent 270 between outer sheath 230 and tubing 287, as depicted in FIG. 6. Alternatively, a solid mandril may be employed in lieu of tubing 287.

Since there is no wire guide lumen depicted in the embodiment of FIGS. 5-6, a shuttle sheath may be used to deliver apparatus 220 to a target site in a patient's vessel. For example, prior to insertion of apparatus 220, a wire guide may be advanced to a desired site, and a shuttle sheath having a diameter larger than the outer diameter of outer sheath 230 may be advanced over the wire guide. In a next step, the wire guide may be removed from the shuttle sheath and apparatus 220 may be distally advanced within the confines of the shuttle sheath. The shuttle sheath then may be removed from the patient's vessel when apparatus 220 is positioned at the target site. Alternatively, a wire guide lumen may be employed, for example, through a longitudinal bore formed in stylet 280 and disc member 282, or through another suitable location.

In the embodiment of FIGS. 5-6, inner tube 240 comprises inner and outer surfaces 247 and 248, respectively, and lumen 243 is formed within the confines of inner surface 247. Fluid that is injected through lumen 243 flows into fluid reservoir 250. During fluid injection, stylet 280, disc member 282 and inner tube 240 may be held longitudinally steady. Optionally, a proximal end of stylet 280 may be coupled to a proximal end of inner tube 240 to allow both components to be advanced, or held steady, simultaneously.

When fluid is injected into fluid reservoir 250 and stylet 280 is held longitudinally steady, distal sealing ring 264 may abut disc member 282, but cannot move distally. Therefore, distal sealing ring 264 provides a fluid-tight seal for fluid reservoir 250 in a distal direction.

As fluid fills fluid reservoir 250 and flows into proximal reservoir section 252, pressure may be imposed upon proximal sealing ring 262. Since outer sheath 230 is not held stationary, the pressure urges sealing ring 262 proximally, which in turn presses upon step 238 of outer sheath 230 to urge outer sheath 230 proximally, as indicated by the arrow in FIG. 6. In effect, as the fluid fills reservoir 250, fluid flowing into proximal reservoir section 252 urges proximal sealing ring 262 and outer sheath 230 in a proximal direction, which in turn exposes stent 270 to enable deployment of the self-expanding stent. By carefully controlling the injection of fluid into lumen 243 and reservoir 250, the physician may incrementally retract outer sheath 230 with respect to inner tube 240.

In the embodiment of FIGS. 3-6, proximal ends of outer sheaths 130 and 230 may terminate a short distance from stents 170 and 270, respectively. For example, as shown in FIG. 5, proximal end 237 of outer sheath 230 terminates just proximal to proximal sealing ring 262 and a relatively short distance from stent 270. Since the physician need not actuate withdrawal of outer sheaths 130 and 230 by pulling on the proximal ends of the sheaths, outer sheaths 130 and 230 need not span a substantial portion of the overall length of inner tubes 140 and 240, respectively.

Advantageously, as noted above, using the hydraulic deployment system of FIGS. 5-6, the longitudinal forces and energy storage imposed upon outer sheath 230 and inner tube 240 may be substantially reduced, relative to techniques that rely on pulling on a proximal end of outer sheath 230 to retract the sheath. Outer sheath 230 may be exposed to less stretching, inner tube 240 may be exposed to less compression, and stent 270 may be less likely to “jump” in a distal direction upon deployment. Accordingly, using apparatus 220, a more accurate deployment of self-expanding stent 270 may be achieved, and the likelihood of the delivery system malfunctioning may be reduced.

As will be apparent, the dimensions of apparatus 220 may be modified to facilitate proximal retraction of outer sheath 230. For example, the dimensions of proximal reservoir section 252 may be increased to provide increased fluid flow to proximal sealing ring 262, which may comprise a greater surface area than depicted in FIGS. 5-6. If proximal sealing ring 262 comprises a greater surface area, it may facilitate retraction of outer sheath 230. Further, the size and configurations of lumens 143 and 243 may be modified to vary the fluid flow into fluid reservoirs 150 and 250, respectively, and/or to vary the force provided upon the proximal sealing rings.

While various embodiments of the invention have been described, it will be apparent to those of ordinary skill in the art that many more embodiments and implementations are possible within the scope of the invention. Accordingly, the invention is not to be restricted except in light of the attached claims and their equivalents. Moreover, the advantages described herein are not necessarily the only advantages of the invention and it is not necessarily expected that every embodiment of the invention will achieve all of the advantages described.

I claim:

1. An apparatus for deploying a self-expanding stent, the apparatus comprising:
an outer sheath comprising proximal and distal regions;
an inner tube comprising proximal and distal regions being disposed substantially coaxially inside of the outer sheath;
a self-expanding stent comprising proximal and distal ends, the self-expanding stent positioned in a radially compressed state within the outer sheath at a location distal to the inner tube;
at least one fluid reservoir formed between the inner tube and the outer sheath; and
a fluid injectable into the fluid reservoir and suitable for imposing a pressure upon the outer sheath to retract the outer sheath when the inner tube is held longitudinally steady.

2. The apparatus of claim 1 further comprising a proximal sealing ring disposed annularly between the inner tube and the outer sheath at a proximal section of the fluid reservoir.

3. The apparatus of claim 2 further comprising a step disposed in the outer sheath at a location adjacent to the proximal sealing ring, wherein proximal advancement of the proximal sealing ring pushes against the step in the outer sheath to thereby cause retraction of the outer sheath.

4. The apparatus of claim 2 further comprising a distal sealing ring disposed annularly between the inner tube and the outer sheath at a distal region of the fluid reservoir.

5. The apparatus of claim 4 further comprising a protruding step formed in the inner tube, wherein the distal sealing ring is disposed proximal to the protruding step such that the distal sealing ring cannot move distally when the inner tube is held longitudinally steady.

6. The apparatus of claim 1 wherein a lumen is formed between inner and outer surfaces of the inner tube, the apparatus further comprising at least one aperture in the outer surface of the inner tube at a location overlying the fluid reservoir to enable fluid communication between the lumen and the fluid reservoir.

7. The apparatus of claim 1 wherein the outer sheath comprises inner and outer members disposed substantially adjacent to one another, the apparatus further comprising a coil member sandwiched between the inner and outer members along a portion of the distal region of the outer sheath.

8. An apparatus suitable for deploying a self-expanding stent, the apparatus comprising:
an outer sheath comprising proximal and distal regions;
a self-expanding stent comprising proximal and distal ends, and further comprising a compressed state and a radially expanded state, wherein the self-expanding stent is adapted to be disposed within the outer sheath and the outer sheath restrains the self-expanding stent in the compressed state;
at least one fluid reservoir disposed adjacent to an interior surface of the outer sheath;
at least one lumen in fluid communication with the fluid reservoir; and
a proximal sealing ring disposed within the outer sheath and disposed proximally from the proximal end of the self-expanding stent,
wherein the delivery of fluid to the fluid reservoir via the lumen is adapted to impose a pressure between the proximal sealing ring and the outer sheath to retract the outer sheath and permit deployment of the self-expanding stent.

9. The apparatus of claim 8 wherein the outer sheath comprises a step disposed adjacent to the proximal sealing ring, such that proximal advancement of the proximal sealing ring pushes against the step in the outer sheath to thereby cause retraction of the outer sheath.

10. The apparatus of claim 8 further comprising an inner tube disposed substantially coaxially inside of the outer sheath, wherein the fluid reservoir is disposed between the inner tube and the outer sheath.

11. The apparatus of claim 10 further comprising a distal sealing ring disposed annularly between the inner tube and the outer sheath within a distal section of the fluid reservoir.

12. The apparatus of claim 11 further comprising a protruding step formed in the inner tube, wherein the distal sealing ring is disposed proximal to the protruding step such that the distal sealing ring cannot move distally when the inner tube is held longitudinally steady.

13. The apparatus of claim 11 further comprising:
a stylet having proximal and distal ends; and
a disc member attached to the distal end of the stylet,
wherein the distal sealing ring is disposed proximal to the disc member and configured to abut the disc member when fluid is disposed in the fluid reservoir.

14. The apparatus of claim 10 wherein the lumen is formed between inner and outer surfaces of the inner tube, the apparatus further comprising at least one aperture disposed in the outer surface of the inner tube at a location overlying the fluid reservoir to enable fluid communication between the lumen of the inner tube and the fluid reservoir.

15. The apparatus of claim 10 wherein the lumen is formed within the inner surface of the inner tube.

16. The apparatus of claim 8 wherein the outer sheath comprises inner and outer members disposed substantially adjacent to one another, the apparatus further comprising a coil member sandwiched between the inner and outer members along a portion of the distal region of the outer sheath.

17. The apparatus of claim 8 wherein the proximal end of the outer sheath terminates just proximal to the self-expanding stent, such that the outer sheath spans less than fifty percent of an overall length of the inner tube.

18. An apparatus suitable for deploying a self-expanding stent, the apparatus comprising:
an outer sheath comprising proximal and distal regions and comprising at least one first threaded member;
an inner tube comprising proximal and distal regions and at least one second threaded member, wherein the inner tube is disposed substantially coaxially inside of the outer sheath; and
a self-expanding stent having proximal and distal ends, and further having a compressed state and a radially expanded state, wherein the self-expanding stent is adapted to be disposed within the outer sheath in the compressed state,
wherein rotation of the first threaded member with respect to the second threaded member is adapted to retract the outer sheath with respect to the inner sheath to permit deployment of the self-expanding stent.

19. The apparatus of claim 18 further comprising:
a protruding step formed in the inner tube and projecting in a radially outward direction, wherein the protruding step is disposed distal to the second threaded member; and
at least one washer disposed annularly between the outer sheath and the inner tube, and further disposed longitudinally between the protruding step of the inner tube and the proximal end of the stent.

20. The apparatus of claim 18 wherein the outer sheath comprises inner and outer members disposed substantially adjacent to one another, and further comprising a coil member sandwiched between the inner and outer members along a portion of the distal region of the outer sheath.
